(19) United States
(12) Patent Application Publication
(10) Pub. No.: US 2010/0255530 A1
(43) Pub. Date: Oct. 7, 2010

(54) BACTERIA DETECTION AND/OR IDENTIFICATION MEDIUM

(75) Inventors: Daniel Monget, Saint Sorlin En Bugey (FR); Sylvain Orenga, Neuville Sur Ain (FR); Michel Peyret, Lyon (FR); Celine Roger-Dalbert, Chazey Sur Ain (FR)

Correspondence Address: OLIFF & BERRIDGE, PLC, P.O. BOX 320850, ALEXANDRIA, VA, 22320-4850, US

(73) Assignee: BIOMERIEUX, MARCY L'ETOILE (FR)

(21) Appl. No.: 12/448,823

(22) PCT Filed: Feb. 7, 2008
(86) PCT No.: PCT/FR2008/050185

§ 371 Date: Jul. 9, 2009

(30) Foreign Application Priority Data
Feb. 8, 2007 (FR) 0753148

(51) Int. Cl. C12Q 1/10

(52) U.S. Cl. 435/38

(57) ABSTRACT

The invention relates to a method for detecting and/or identifying E. coli in a urine sample, that comprises:
a) inoculating the urine sample liable to contain E. coli on a detection medium that comprises
   a first substrate selected from a beta-glucuronidase substrate, beta-galactosidase substrate and alpha-galactosidase substrate, and a substrate for a lactose acidification enzyme, beta-ribosidase, phosphatase, L-alanine aminopeptidase and L-leucine aminopeptidase, and
   a second substrate, different from said first substrate and selected from a beta-glucuronidase substrate, beta-galactosidase substrate and alpha-galactosidase substrate, and a substrate for a lactose acidification enzyme, beta-ribosidase, phosphatase, L-alanine aminopeptidase and L-leucine aminopeptidase,
   or obtaining bacterial colonies;
b) identifying the colonies that react with the first substrate and/or the second substrate as being colonies of E. coli.

The field of the invention is that of biochemical microbiological analysis, and in particular of the detection and identification of bacteria.

Pathogenic bacteria, and in particular Gram-negative bacilli, such as enterobacteria, are responsible each year for many diseases, epidemics, etc.

The species E. coli (Escherichia coli) is the aerobic species most predominantly represented in the digestive tract. However, the presence of said bacteria in water indicates fecal contamination, and certain strains are pathogenic and responsible for peritoneal, biliary, appendicular or genital suppurations.

Early and specific detection of E. coli makes it possible to propose a suitable solution, in terms of treatment, of decontamination, etc. This detection can be based in particular on the use of detection media comprising particular substrates, specific for a metabolic activity, referred to as target metabolic activity, such as an enzymatic activity, of the bacterium that it is desired to detect: through the choice of substrates, depending on whether or not there is a reaction, it is possible to characterize the nature of a microorganism.

The CPS ID 3 (bioMerieux) medium uses a β-glucuronidase substrate combined with a β-glucosidase substrate and, optionally, with the detection of tryptophanase, for detecting strains of the Escherichia coli species. However, while this medium has excellent specificity, the use of a β-glucuronidase substrate for detecting E. coli exhibits imperfect sensitivity owing to the existence of a small proportion of E. coli strains (5-10%) which do not express this activity. Furthermore, certain Citrobacter strains can also produce β-glucuronidase-positive colonies that are the same color as those of E. coli.

The invention proposes to solve the prior art problems by providing a new medium that is particularly suitable for identifying E. coli bacteria rapidly and inexpensively and in a manner that is easy to implement. Surprisingly, the inventors have shown that a particular combination of enzymatic substrates, at suitable concentrations, enables rapid and easy detection of E. coli, in particular using a urine sample.

Before proceeding with the disclosure of the invention, the following definitions are given in order to facilitate the understanding of the invention.

The term detection medium is intended to mean a medium comprising all the elements necessary for the survival and/or the growth of microorganisms. This detection medium can either serve as detection medium only, or as culture and detection medium. In the first case, the culturing of the microorganisms is carried out before inoculation, and in the second case, the detection medium also constitutes the culture medium. The culture medium according to the invention may contain other possible additives, for instance: peptones or extracts of tissues, one or more growth factors, carbohydrates, one or more selective agents, buffers, one or more gelling agents, etc. This culture medium may be in liquid form or in the form of a ready-to-use gel, ready for seeding in a tube or flask or on a Petri dish.

For the purpose of the present invention, the detection can be carried out in liquid medium, a strip, or another solid support.

The term substrate is intended to mean any molecule capable of directly or indirectly generating a detectable signal due to an enzymatic or metabolic activity of the microorganism.

The substrate may in particular be a metabolic substrate, such as a carbon or nitrogen source, coupled to an indicator that produces a coloration in the presence of one of the products of the metabolism.

The substrate may also be an enzymatic substrate, a substrate that can be hydrolyzed by an enzyme so as to give a product that enables direct or indirect detection of a microorganism. This substrate may in particular comprise a first part which is specific for the enzymatic activity to be revealed and a second part which acts as a label, hereinafter known as label part. This label part may be chromogenic, fluorogenic, luminescent, etc. As a chromogenic substrate suitable for solid supports (filter, agar, electrophoresis gel), mention may in particular be made of substrates based on indoxyl and its derivatives, and substrates based on hydroxyquinoline or on esculetin and their derivatives, which enable the detection of osidase and esterase activities. Mention may also be made of substrates based on nitrophenol and nitroaniline and derivatives, for detecting osidase and esterase activities in the case of nitrophenol-based substrates, and peptidase activities in the case of nitroaniline-based substrates. Finally, mention may be made of substrates based on naphthol and naphthylamine and their derivatives, which make it possible to detect osidase and esterase activities by means of naphthol, and peptidase activities by means of naphthylamine. This substrate may in particular, but in a nonlimiting manner, enable the detection of an enzymatic activity such as the activity of an osidase, peptidase, esterase, etc. The enzymatic substrate may also be a natural substrate, the product of hydrolysis of which is detected directly or indirectly. As a natural substrate, mention may in particular be made of tryptophan for detecting a tryptophanase or deaminase activity, a cyclic amino acid (tryptophan, phenylalanine, histidine, tyrosine) for detecting a deaminase activity, phosphatidylinositol for detecting a phospholipase activity, etc.

According to the present invention, the substrate is preferably selected from substrates based on indoxyl (3-indoxyl, 5-bromo-3-indoxyl, 5-iodo-3-indoxyl, 4-chloro-3-indoxyl, 5-bromo-4-chloro-3-indoxyl, 5-bromo-6-chloro-3-indoxyl, 6-bromo-3-indoxyl, 6-chloro-3-indoxyl, 6-fluoro-3-indoxyl, 5-bromo-4-chloro-N-methyl-3-indoxyl, N-methyl-3-indoxyl, etc.); on umbelliferone (4-methylumbelliferone, cyclohexenoesculetin, etc.); on alizarine; on p-naphtholbenzein; on nitrophenol (ortho-nitrophenol, para-nitrophenol, etc.); on hydroxyquinoline; on cathechol (cathecol, dihydroxyflavone, hydroxyflavone, etc.); on resorufin; on Chlorophenol Red; on fluorescein; on aminophenol (para-aminophenol, dichloroaminophnol, etc.); on naphthol (alpha-naphthol, 2-naphthol, Naphthol-ASBI, etc.); on aminocoumarin (7-amino-4-methylcoumarin, etc.); on naphthylamide; on acridine (aminophenylacridine, etc.); or on aminophenoxazine (aminobenzophenoxazinone, aminopentylresorufin, etc.).

By way of indication, the substrates used for detecting a beta-glucuronidase activity may in particular be 4-methylumbelliferyl-beta-glucuronide, 5-bromo-4-chloro-3-indolyl-beta-glucuronide, 5-bromo-6-chloro-3-indolyl-beta-glucuronide, 6-chloro-3-indolyl-beta-glucuronide, alizarine-beta-glucuronide or cyclohexenoesculetin-beta-glucuronide, or salts thereof.

The substrates used for detecting a beta-galactosidase activity may in particular be 4-methylumbelliferyl-beta-galactoside, 5-bromo-4-chloro-3-indolyl-beta-galactoside, 5-bromo-6-chloro-3-indolyl-beta-galactoside, 6-chloro-3-indolyl-beta-galactoside, alizarine-beta-galactoside or cyclohexenoesculetin-beta-galactoside, or salts thereof.

The substrates used for detecting a beta-glucosidase activity may in particular be 4-methylumbelliferyl-beta-glucoside, 5-bromo-4-chloro-3-indolyl-beta-glucoside, 5-bromo-6-chloro-3-indolyl-beta-glucoside, 6-chloro-3-indolyl-beta-glucoside, alizarine-beta-glucoside, cyclohexenoesculetin-beta-glucoside, nitrophenyl-beta-glucoside or dichloroaminophenylglucoside. or salts thereof.

The term inducer is intended to mean a compound which induces an increase in the expression of the targeted metabolic activity; all experimental conditions being otherwise equal, the metabolic activity is greater when the inducer is at appropriate concentration than when it is absent or at an unsuitable concentration.

Without being limiting, a concentration of between 100 ng/l and 10 g/l, preferably between 10 mg/l and 3 g/l, is particularly suitable for the present invention.

Mention may in particular be made of:
for beta-glucuronidase, a glucuronide preferably selected from glucuronate and methyl-beta-glucuronide;
for beta-galactosidase, a galactoside preferably selected from lactose and isopropyl-beta-thio galactoside;
for beta-glucosidase, a carbohydrate constituted of a carbohydrate linked in the β-position to glucose, or a carbohydrate with a β-glucoside subunit, in particular cellobiose, cellulose, starch, cellotriose or trehalose. Mention may also be made of methyl-β-glucoside, isopropyl-β-thioglucoside, indoxyl-β-glucoside or methyl-β-thioglucoside.

The term inhibitor is intended to mean a compound which induces a decrease in the expression of the targeted metabolic activity; all experimental conditions being otherwise equal, the metabolic activity is weaker when the inducer is at an appropriate concentration than when it is absent or at an unsuitable concentration. Without being limiting, a concentration of between 100 ng/l and 30 g/l, preferably between 1 mg/l and 3 g/l, is particularly suitable for the present invention.

Mention may in particular be made of
for beta-glucuronidase: D-glucose, D-glucaric acid 1,4-lactone
for beta-galactosidase: 2-deoxygalactose, cellobiose, D-galactose, D-glucose.

The term biological sample is intended to mean clinical sample, derived from a sample of biological fluid, or a food sample, derived from any type of food, or an environmental sample such as a surface sample, water sample, air sample. etc. This sample may thus be liquid or solid and mention may be made, in a nonlimiting manner, of a clinical sample from blood, plasma, urine or feces, samples taken from the nose, from the throat, from the skin, from wounds or from cerebrospinal fluid, a food sample from water, or from drinks such as milk or a fruit juice; from yoghurt, meat, eggs, vegetables, mayonnaise or cheese; from fish, etc., or a food sample derived from an animal feed, such as in particular a sample derived from animal meals.

In this respect, the invention relates to a method for detecting and/or identifying E. coli in a biological sample, preferably a urine sample, that comprises;
a) inoculating the sample, preferably urine sample, liable to contain E. coli on a detection medium that comprises
   a first substrate selected from a beta-glucuronidase substrate, beta-galactosidase substrate and alpha-galactosidase substrate, and a substrate for a lactose acidification enzyme, beta-ribosidase, phosphatase, L-alanine aminopeptidase and L-leucine aminopeptidase, and
   a second substrate, different from said first substrate and selected from a beta-glucuronidase substrate, beta-galactosidase substrate and alpha-galactosidase substrate, and a substrate for a lactose acidification enzyme, beta-ribosidase, phosphatase, L-alanine aminopeptidase and L-leucine aminopeptidase,
   for obtaining bacterial colonies;
b) identifying the colonies that react with the first substrate and/or the second substrate as being colonies of E. coli.

Preferably, said first and second substrates are at a suitable concentration. The inoculation of the microorganisms can be carried out by any of the inoculation techniques known to those skilled in the art. An incubation step may be carried out at a temperature for which the enzymatic activity that it is desired to detect is optimal, it being possible for those skilled in the art to readily select said temperature according to the enzymatic activity to be detected. The detection/identification can be carried out by means of a visual examination, by colorimetry or fluorimetry.

According to one preferred embodiment of the invention, said first substrate is at a concentration of between 20 and 1000 mg/l and said second substrate is at a concentration of between 20 mg/ and 30 g/l.

According to one preferred embodiment of the invention, said first substrate is a beta-glucuronidase substrate and the second substrate is a beta-galactosidase substrate. Preferably, the substrate for beta-glucuronidase activity is selected from 4-methylumbelliferyl-beta-glucuronide, 5-bromo-4-chloro-3-indolyl-beta-glucuronide, 5-bromo-6-chloro-3-indolyl-beta-glucuronide, 6-chloro-3-indolyl-beta-glucuronide, alizarine-beta-glucuronide or cyclohexenoesculetin-beta-glucuronide, or salts thereof, at concentrations of preferably between 20 and 1000 mg/l.

Preferably, the beta-galactosidase substrate is at a low concentration. Preferably, the substrate for beta-galactosidase activity is selected from 4-methylumbelliferyl-beta-galactoside, 5-bromo-4-chloro-3-indolyl-beta-galactoside, 5-bromo-6-chloro-3-indolyl-beta-galactoside, 6-chloro-3-indolyl-beta-galactoside, alizarine-beta-galactoside or cyclohexenoesculetin-beta-galactoside, or salts thereof, at a concentration preferably of between 10 and 1000 mg/l, preferably between 20 and 500 mg/l.

According to one preferred embodiment of the invention, the detection medium also comprises a third substrate, preferably selected from a substrate for beta-glucosidase, beta-lactosidase, N-acetylhexosaminidase, esterase, sulfatase, beta-xylosidase, phospholipase, alpha-mannosidase, beta-mannosidase, beta-cellobiosidase, alpha-glucosidase, tryptophanase, deaminase, oxydase, pigment synthesis, peptidases (beta-alanine aminopeptidase, elastase, etc.).

Preferably, said third substrate is a substrate for beta-glucosidase. Preferably, the substrate for beta-glucosidase activity is selected from 4-methylumbelliferyl-beta-glucoside, 5-bromo-4-chloro-3-indolyl-beta-glucoside, 5-bromo-6-chloro-3-indolyl-beta-glucoside, 6-chloro-3-indolyl-beta-glucoside, cyclohexenoesculetin-beta-glucoside, nitrophenyl-beta-glucoside or dichloroaminophenylglucoside, or salts thereof, at a concentration preferably of between 10 and 1000 mg/l, preferably between 20 and 500 mg/l. According to one preferred embodiment of the invention, the detection medium also comprises an inducer.

Preferably, the inducer is at a concentration of between 100 ng/l and 10 g/l.

According to one preferred embodiment of the invention, the inducer is preferably:
for beta-glucuronidase, a glucuronide preferably selected from glucuronate and methyl-beta-glucuronide;
for beta-galactosidase, a galactoside preferably selected from lactose and isopropyl-beta-thiogalactoside;
for beta-glucosidase, a carbohydrate constituted of a carbohydrate linked in the β-position to glucose, or a carbohydrate with a β-glucoside subunit, in particular cellobiose, cellulose, starch, cellotriose or trehalose. Mention may also be made of methyl-β-glucoside, isopropyl-β-thioglucoside, indoxyl -β-glucoside or methyl-β-thioglucoside.

Preferably, the inducer is cellobiose, at a concentration preferably of between 100 ng/l and 10 g/l.

According to one preferred embodiment of the invention, the detection medium also comprises an inhibitor. Preferably, the inhibitor is at a concentration of between 100 ng and 30 g/l.

According to one preferred embodiment of the invention, the inhibitor is preferably:
for beta-glucuronidase: D-glucose, D-glucaric acid 1,4-lactone
for beta-galactosidase: 2-deoxygalactose, cellobiose, D-galactose, D-glucose.

The examples below are given by way of explanation and are in no way limiting in nature. They will make it possible to understand the invention more clearly.

### EXAMPLE 1

### Evaluation of the combination of 6-chloro-3-indolyl-β-glucuronide and 5-bromo-6-chloro-3-indolyl-β-galactoside

Various concentrations of 6-chloro-3-indolyl-β-glucuronide (0-0.1-0.15 and 0.20 g/l) and of 5-bromo-6-chloro-3-indolyl-β-galactoside (0-0.025-0.05 and 0.1 g/l) are added to and combined with the CPS ID 3 medium (bioMerieux) from which the synthetic enzymatic substrate for β-glucuronidase has been removed. These media also comprise 5-bromo-4-chloro-3-indolyl-β-glucoside at 50 mg/l. They are distributed in a proportion of 20 ml per Petri dish. The Coli ID medium (bioMerieux) which combines a β-glucuronidase substrate (6-chloro-3-indolyl-β-glucuronide) and a β-galactosidase substrate (5-bromo-3-indolyl-β-galactoside), intended for the detection and counting of E. coli and coliforms in food samples, is tested in parallel. Microorganisms commonly isolated from urine samples and derived from the applicant's collection are inoculated onto these media by semi-quantitative isolation of 10 μl of a suspension at 0.5 McFarland, diluted to 1/20. The dishes are incubated at 37° C. for 20 hours, and then the colonies formed are examined visually. The color of these colonies is noted. The results are given in table 1 below:

TABLE 1
Impact of the combination of 6-chloro-3-indolyl-β-glucuronide and 5-bomo-6-chloro-
3-indolyl-β-galactoside in the CPS ID 3 medium on colony coloration

| | Medium No. | | | | | | | | | | | | |
|---|---|---|---|---|---|---|---|---|---|---|---|---|---|
| | 1b | 1c | 2a | 2b | 2c | 3a | 3b | 3c | 4 | 4a | 4b | 4c | Coli ID |
| [6-Chloro-3-indolyl- | 0 | 0 | 0.1 | 0.1 | 0.1 | 0.15 | 0.15 | 0.15 | 0.2 | 0.2 | 0.2 | 0.2 | 0.2 |
| β-glucuronide] in g/l | | | | | | | | | | | | | |
| [5-Bromo-6-chloro- | 0.05 | 0.1 | 0.025 | 0.05 | 0.1 | 0.025 | 0.05 | 0.1 | 0 | 0.025 | 0.05 | 0.1 | NA |
| 3-indolyl-β-galactoside] in | | | | | | | | | | | | | |
| g/l | | | | | | | | | | | | | |
| Escherichia coli 206 | P | P | P | P | P | P | P | P | P | P | P | P | P |
| Escherichia coli 067 | — | — | — | — | Pp | — | Pp | Pp | — | — | Pp | Pp | GB |
| Escherichia coli 037 | Pp | P | P | P | P | P | P | P | P | P | P | P | P |
| Citrobacter freundii 064 | P | P | — | P | P | — | P | P | — | — | P | P | B |
| Citrobacter diversus 097 | GG | GP | GG | GG | GP | GG | GG | GP | GG | GG | GG | GP | B |
| Citrobacter diversus 010 | GG | GP | GG | GG | GP | GG | GG | GP | GG | GG | GG | GP | B |
| Klebsiella pneumoniae 023 | BG | BG | BG | BG | BG | BG | BG | BG | BG | BG | BG | BG | B |
| Enterobacter cloacae 059 | GP | Vi | BG | GP | Vi | GG | GP | Vi | BG | GG | GP | Vi | B |
| Proteus mirabilis 054 | O | O | O | O | O | O | O | O | O | O | O | O | — |
| Enterococcus faecalis 117 | T | T | T | T | T | T | T | T | T | T | T | T | Inh |

NA = not applicable,
—= colorless,
Inh = inhibited,
P = pink,
Pp = pale pink,
GP = grayish-pink,
GG = grayish-green,
GB = grayish-blue,
BG = bluish-green,
Vi = violet,
O = orangey-brown,
T = turquoise

In table 1, it emerges that only the media 2c, 3b, 3c, 4b and 4c combining 6-chloro-3-indolyl-β-glucuronide with 5-bromo-6-chloro-3-indolyl-β-galactoside make it possible to detect all the E. coli strains. This is not the case of the Coli ID medium, which nevertheless combines a β-glucuronidase substrate and a β-galactosidase substrate. However, on the media 2c, 3c and 4c, the E. cloacae strain is less readily distinguished from the E. coli strains. Similarly, the C. freundii strain produces colonies of the same color as the E. coli strains on all the media having at least 0.05 g/l of 5-bromo-6-chloro-3-indolyl-β-galactoside.

Thus, it is possible to determine the media that are the most advantageous for improving the sensitivity of detection of E. coli strains without being too damaging to the specificity.

### EXAMPLE 2

### Impact of cellobiose on a medium combining 6-chloro-3-indolyl-β-glucuronide, 5-bromo-6-chloro-3-indolyl-β-galactoside and 5-bromo-4-chloro-3-indolyl-β-glucoside

6-Chloro-3-indolyl-β-glucuronide, 5-bromo-6-chloro-3-indolyl-β-galactoside and 5-bromo-4-chloro-3-indolyl-β-glucoside are added, at 0.15 g/l, 0.08 g/l and 0.08 g/l respectively, to Trypticase Soya Agar Medium (bioMerieux). This medium is supplemented, or not supplemented, with cellobiose at 0.5 g/l. These two media are distributed in a proportion of 20 ml per Petri dish. Microorganisms commonly isolated from urine samples and derived from the applicant's collection are inoculated onto these media by semi-quantitative isolation of 10 μl of a suspension at 0.5 McFarland, diluted to 1/20. The dishes are incubated at 37° C. for 24 hours, and then the colonies formed are examined visually. The coloration of these colonies is noted. The results are given in table 2 below:

TABLE 2
Impact of cellobiose on a medium combining 6-chloro-3-indolyl-
β-glucuronide, 5-bromo-6-chloro-3-indolyl-β-galactoside and
5-bromo-4-chloro-3-indolyl-β-glucoside with
respect to colony coloration

Concentration of
cellobiose in mg/l

| Strains | 0 | 500 |
|---|---|---|
| Escherichia coli 407 | pink | pink |
| Escherichia coli 067 | pink | pink |
| Klebsiella pneumoniae 111 | turquoise | turquoise |
| Serratia marcescens 112 | turquoise | turquoise |
| Citrobacter freundii 031 | gray | gray |
| Citrobacter freundii 009 | pink | violet |
| Streptococcus agalactiae 019 | mauve | mauve |
| Enterococcus faecalis 117 | turquoise | turquoise |

In table 2 above, it emerges that, in a medium combining 6-chloro-3-indolyl-β-glucuronide and 5-bromo-6-chloro-3-indolyl-β-galactoside, cellobiose makes it possible to distinguish the Citrobacter 009 strain more clearly from the E. coli strains. This makes it possible to benefit from the gain in sensitivity for the detection of E. coli without being penalized by damage to the specificity.

### EXAMPLE 3

### Test for Defining the Concentration of Said First and Second Substrates According to the Invention

The test below can be carried out in order to define the concentration of said first and second substrates according to the invention, which is variable depending on the substrates used and, more generally, on the formulation of the reaction medium. In order to aid the understanding of this test, it is carried out below in the case of a combination of β-glucuronidase and β-galactosidase, using a kit of microorganism strains, comprising in particular E. coli strains, including strains which do not express a positive activity or which express a positive activity weakly or late, and optionally other microorganisms. This test can be carried out for other types of substrates. Two reaction media comprising either a suitable concentration of β-glucuronidase substrate or no β-glucuronidase substrate are used to prepare a β-galactosidase substrate range including a zero concentration, at least one concentration for obtaining a positive reaction with the E. coli strains expressing a β-galactosidase, and also intermediate concentrations. Each of the media is aliquoted in such a way that each microorganism strain can be inoculated in culture, pure, on each of the media. After a suitable incubation time, generally between 30 minutes and 72 hours, at an appropriate temperature of preferably between 20 and 50° C., the media are examined so as to select the medium comprising a combination of substrates for β-galactosidase and for β-glucuronidase that makes it possible to reveal the greatest number of E. coli strains while at the same time distinguishing them from the greatest number of strains of the other microorganisms. It may be necessary to repeat the experiment with the concentrations of each of the substrates and also the strain kit being adjusted. It may be advantageous for the reaction media to also comprise inducers and/or inhibitors of β-galactosidase and/or β-glucuronidase.

1. A method for detecting and/or identifying E. coli in a urine sample, that comprises:
a) inoculating the urine sample liable to contain E. coli on a detection medium that comprises
a first substrate selected from a beta-glucuronidase substrate, beta-galactosidase substrate and alpha-galactosidase substrate, and a substrate for a lactose acidification enzyme, beta-ribosidase, phosphatase, L alanine aminopeptidase and L-leucine aminopeptidase, and
a second substrate, different from said first substrate and selected from a beta-glucuronidase substrate, beta-galactosidase substrate and alpha-galactosidase substrate, and a substrate for a lactose acidification enzyme, beta-ribosidase, phosphatase, L-alanine aminopeptidase and L-leucine aminopeptidase, for obtaining bacterial colonies;
b) identifying the colonies that react with the first substrate and/or the second substrate as being colonies of E. coli.

2. The method as claimed in claim 1, according to which the first substrate is a beta-glucuronidase substrate and the second substrate is a beta-galactosidase susbtrate.

3. The method as claimed in claim 2, according to which said first substrate is at a concentration of between 20 and 1000 mg/l and the second substrate is at a concentration of between 10 mg/ and 30 g/l

4. The method as claimed in claim 1, according to which the detection medium also comprises a third substrate selected from a substrate for beta-glucosidase, beta-lactosidase, N-acetylhexosaminidase, esterase, sulfatase, beta-xylosidase, phospholipase, alpha-mannosidase, beta-mannosidase, beta-cellobiosidase, alpha-glucosidase, tryptophanase, deaminase, oxydase, pigment synthesis, peptidases (beta-alanine aminopeptidase, elastase, etc.).

5. The method as claimed in claim 4, according to which the third substrate is a beta glucosidase substrate.

6. The method as claimed in claim 1, according to which the detection medium also comprises an inducer.

7. The method as claimed in claim 1, according to which the detection medium also comprises an inhibitor.
